(19) United States
(12) Patent Application Publication
(10) Pub. No.: US 2023/0293809 A1
(43) Pub. Date: Sep. 21, 2023

(54) ELECTRONIC PUMP ASSEMBLY FOR AN IMPLANTABLE DEVICE HAVING AN ACTIVE VALVE

(71) Applicant: Boston Scientific Scimed, Inc., Maple Grove, MN (US)

(72) Inventor: Eduardo Marcos Larangeira, Cork City (IE)

(21) Appl. No.: 18/182,607

(22) Filed: Mar. 13, 2023

Related U.S. Application Data
(60) Provisional application No. 63/269,447, filed on Mar. 16, 2022.

(51) Int. Cl. A61M 5/142

(52) U.S. Cl. CPC A61M 5/142; A61M 2205/3317; A61M 2205/3331

(57) ABSTRACT

According to an aspect, an active valve for an implantable device includes a base plate defining an opening, a piezo element, a diaphragm actuator coupled to the piezo element, and a protrusion coupled to the diaphragm actuator. The diaphragm actuator, in response to the piezo element being activated, is configured to move the protrusion into the opening in a first direction until the protrusion contacts a portion of the base plate.

### CROSS-REFERENCE TO RELATED APPLICATION

This application claims priority to U.S. Provisional Patent Application No. 63/269,447, filed on Mar. 16, 2022, entitled “AN ELECTRONIC PUMP ASSEMBLY FOR AN IMPLANTABLE DEVICE HAVING AN ACTIVE VALVE”, the disclosure of which is incorporated by reference herein in its entirety.

### TECHNICAL FIELD

This disclosure relates generally to bodily implants and more specifically to bodily implants, such as electronic implantable devices having an active valve.

### BACKGROUND

An implantable device may include a valve to permit or block the flow of fluid between components of the implantable device. In some examples, a conventional valve may not provide a sufficient gap to allow a desired flow rate while minimizing leakage through the valve.

### SUMMARY

According to an aspect, an active valve for an implantable device includes a base plate defining an opening, a piezo element, a diaphragm actuator coupled to the piezo element, and a protrusion coupled to the diaphragm actuator. The diaphragm actuator, in response to the piezo element being activated, is configured to move the protrusion into the opening in a first direction until the protrusion contacts a portion of the base plate.

The active valve may include one or more of the following features (or any combination thereof). The protrusion includes a tapered conical portion. The protrusion includes a metal-based material. The opening includes a tapered conical hole. The diaphragm actuator includes a metal-based material. The diaphragm actuator is coupled to the base plate. The diaphragm actuator includes a first surface and a second surface, the piezo element being coupled to the first surface of the diaphragm actuator, the protrusion being coupled to the second surface of the diaphragm actuator. The diaphragm actuator includes a metal-based material. At least a portion of the protrusion is disposed outside of the opening of the base plate in response to the piezo element not being actuated.

According to an aspect, an implantable device includes a fluid reservoir configured to hold fluid, an inflatable member, and an electronic pump assembly including a controller and an active valve. The active valve includes a base plate defining an opening, a piezo element, a diaphragm actuator coupled to the piezo element, and a protrusion coupled to the diaphragm actuator. The controller is configured to activate the piezo element to move the protrusion into the opening in a first direction until the protrusion contacts a portion of the base plate. The controller is configured to controller is configured to deactivate the piezo element to move at least a portion of the protrusion out of the opening. The protrusion includes a metallic needle portion. The metallic needle portion includes a tapered conical portion. The base plate includes a metal-based material, and the opening on the base plate includes a tapered conical hole configured to receive the tapered conical portion. The diaphragm actuator is welded to the base plate. The piezo element is coupled to the diaphragm actuator with an epoxy-based material. The opening is an inlet port configured to receive fluid, and the base plate defines an outlet port to output fluid. The inlet port is coupled to the inflatable member and the outlet port is coupled to the fluid reservoir.

According to an aspect, a method for actuating an active valve of an implantable device includes receiving a first control signal to apply a voltage to a piezo element of an active valve of an implantable device, the active valve including a base plate defining an opening, a diaphragm actuator coupled to the piezo element, and a protrusion coupled to the diaphragm actuator, and moving, in response to actuation of the piezo element, the protrusion into the opening of the base plate in a first direction until the protrusion contacts a portion of the base plate. In some examples, the method includes receiving a second control signal to not apply the voltage to the piezo element of the active valve and moving, in response to the second control signal, the protrusion in a second direction such that at least a portion of the protrusion is disposed outside of the opening of the base plate. In some examples, the method includes forming a metal-to-metal seal with the protrusion and the base plate.

### BRIEF DESCRIPTION OF THE DRAWINGS

FIG. 1A illustrates an implantable device having an active valve according to an aspect.

FIG. 1B illustrates an example of the active valve according to an aspect.

FIG. 2A illustrates an example of a diaphragm actuator and a protrusion of the active valve according to an aspect.

FIG. 2B illustrates an example of a base plate and the diaphragm actuator according to an aspect.

FIG. 2C illustrates an example of the diaphragm actuator coupled to the base plate according to an aspect.

FIG. 2D illustrates an example of a piezo element coupled to the diaphragm actuator according to an aspect.

FIG. 2E illustrates an example of an active valve in an open position according to an aspect.

FIG. 3 illustrates a flowchart depicting example operations of an active valve according to an aspect.

FIG. 4 illustrates a flowchart depicting example operations of manufacturing an active valve according to an aspect.

FIG. 5 illustrates an example of an inflatable penile prosthesis according to an aspect.

FIG. 6 illustrates an example of an artificial urinary sphincter device according to an aspect.

### DETAILED DESCRIPTION

This disclosure relates to an active valve that can improve the performance of an implantable device. For example, the active valve discussed herein may achieve a relatively consistent flow rate (over a period of time) while minimizing the leakage through the active valve. The active valve may include a piezoelectric diaphragm valve. The active valve is configured to transition from an open position (in which fluid is permitted through the active valve) and a closed position (in which fluid is blocked from being transferred through the active valve). In some examples, in the closed position, the active valve provides a metal-to-metal seal to block the transfer of fluid through the active valve. In some examples, the metal-to-metal seal may provide consistent performance (e.g., given that metal machining may allow tighter tolerances when compared to higher tolerances required by rubber-based materials). In some examples, the active valve does not include a rubber-based material (e.g., a rubber-based ring member or O-ring) to provide the sealing, which may minimize the risk of adding particles to the fluid (e.g., saline) (e.g., most rubber-based materials are likely to wear out during operation).

The active valve may include a diaphragm actuator coupled to a protrusion. The diaphragm actuator may include a metal-based material. In some examples, the diaphragm actuator may be a thin, flexible piece (e.g., circular/cylindrical piece) of a metal material (e.g., foil). In some examples, the protrusion is a metallic needle portion. In some examples, the metallic needle portion includes a tapered conical portion. The protrusion may be welded to a surface (e.g., a central portion) of the diaphragm actuator. The active valve includes a base plate defining an inlet port and an outlet port. When the active valve is in the open position, fluid may be transferred from the inlet port to the outlet port. When the active valve is in the closed position, fluid may be blocked from being transferred from the inlet port to the outlet. The base plate may be a metal cylindrical portion. The inlet port is an opening (e.g., a tapered conical hole) configured to receive the protrusion (to block the flow). For example, the base plate is coupled (e.g., welded) to the diaphragm actuator such that the protrusion is aligned with the opening. The active valve includes a piezo element coupled to the diaphragm actuator (e.g., via an epoxy material). In some examples, the piezo element is a structure that is configured to change the element's shape when a voltage is applied.

In response to the piezo element being activated (e.g., a voltage applied to the piezo element), the diaphragm actuator moves towards the base plate such that the protrusion moves into the opening of the base plate in a first direction until the protrusion contacts a portion of the base plate (within the opening), thereby providing a metal-to-metal sealing. In response to the piezo element being deactivated (e.g., a voltage being removed from the piezo element), the diaphragm actuator moves away from the base plate such that at least a portion of the protrusion is disposed outside of the opening, thereby allowing the fluid to flow through the active valve.

In some examples, the implantable device is an artificial urinary sphincter device. In some examples, the implantable device is an inflatable penile prosthesis. An implantable device may include an inflatable member, an electronic pump assembly, and a fluid reservoir. The electronic pump assembly may transfer fluid between the fluid reservoir and the inflatable member without the user manually operating a pump bulb. For example, the transfer of fluid between the inflatable member and the fluid reservoir is electrically controlled.

The electronic pump assembly may include a controller, one or more active valves, one or more pumps, and one or more pressure sensors. The electronic pump assembly may include circuit board(s), batteries, and communication module(s) to control the inflatable member. The controller (e.g., processor(s), driver(s)) may control (e.g., actuate, activate, deactivate, move, etc.) the pump(s) and the active valve(s) to move fluid between the inflatable member and the fluid reservoir to transition the inflatable member between an inflation state and a deflation state. In some examples, the pump(s) are activated to transfer fluid to the inflatable member and an active valve is disposed in a fluid passageway that is used to empty the inflatable member. In some examples, the controller may cause the active valve to move to an open position to deflate the inflatable member. In some examples, the active valve is transitioned to an open position to transfer fluid from the fluid reservoir to the inflatable member during an inflation cycle, and the pump(s) are activated to transfer fluid from the inflatable member to the fluid reservoir in a deflation cycle.

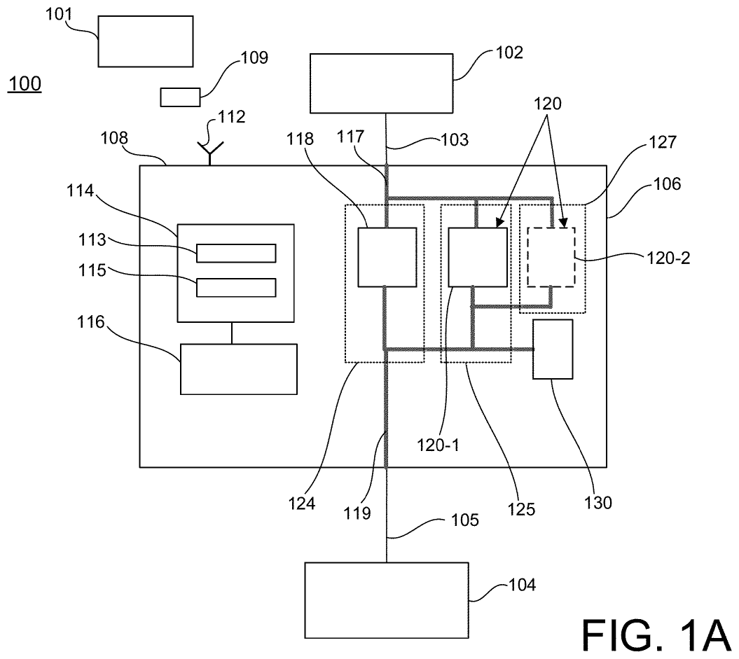

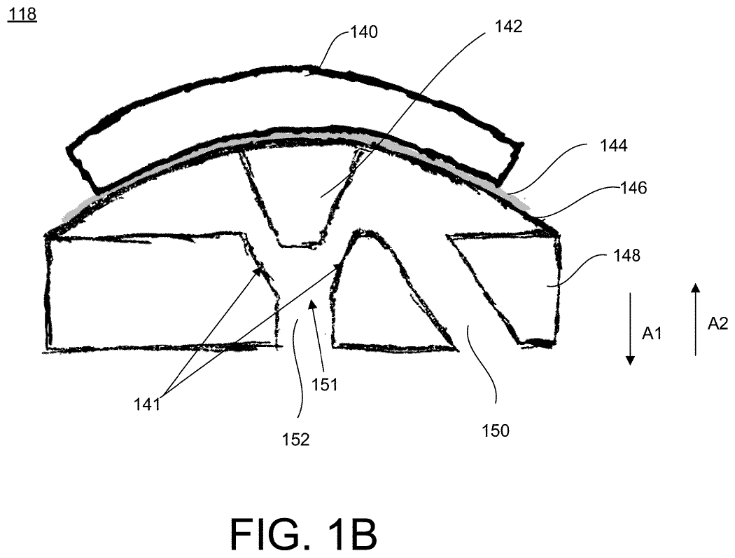

FIG. 1A illustrates an implantable device 100 according to an aspect. The implantable device 100 may include a fluid reservoir 102, an inflatable member 104, and an electronic pump assembly 106 configured to transfer fluid between the fluid reservoir 102 and the inflatable member 104. In some examples, the implantable device 100 is an artificial urinary sphincter device. In some examples, the implantable device 100 is an inflatable penile prosthesis. However, the implantable device 100 may include any type of medical device that transfers fluid between components of the implantable device 100.

FIG. 1B illustrates an example of an active valve 118. One of more active valves 118 may be included in the electronic pump assembly 106. The active valve 118 may be an electronically-controlled valve. The active valve 118 may be electronically-controlled by a controller 114 of the electronic pump assembly 106. The active valve 118 may achieve a desired flow rate (over a period of time) while minimizing the leakage through the active valve 118. In some examples, the active valve 118 includes a piezoelectric diaphragm valve. The active valve 118 is configured to transition from an open position (in which fluid is permitted through the active valve 118) and a closed position (in which fluid is blocked from being transferred through the active valve 118).

In some examples, in the closed position, the active valve 118 provides a metal-to-metal seal to block the transfer of fluid through the active valve 118. In some examples, the metal-to-metal seal may provide consistent performance (e.g., given that metal machining may allow tighter tolerances when compared to higher tolerances required by rubber-based materials). In some examples, the active valve 118 does not include a rubber-based material (e.g., a rubber-based ring member or O-ring) to provide the sealing, which may minimize the risk of adding particles to the fluid (e.g., saline) (e.g., most rubber-based materials are likely to wear out during operation).

Referring to FIG. 1A, the electronic pump assembly 106 may include a single active valve 118. In some examples, the electronic pump assembly 106 includes multiple active valves 118. In some examples, one or more additional active valves 118 may be in series with a pump 120-1 and/or a pump 120-2. In some examples, an additional active valve 118 (e.g., a series active valve 118) may be disposed in a fluid pathway portion 117 that is connected to the fluid reservoir 102. In some examples, an additional active valve 118 (e.g., a series active valve 118) may be disposed in a fluid pathway portion 119 that is connected to the inflatable member 104. These additional active valves 118 may reduce leakage when at maximum inflation pressure or at partial inflation pressure.

Referring to FIG. 1A, the active valve 118 may be connected to the controller 114 of the electronic pump assembly 106 and may receive a signal to transition the active valve 118 between the open position and the closed position. In some examples, the active valve 118 is disposed in a fluid passageway 124 that is used to empty the inflatable member 104 (e.g., in the deflation cycle). In some examples, the active valve 118 is disposed in a fluid passageway 124 that is used to fill the inflatable member 104 (e.g., in the inflation cycle). In some examples, the active valve 118 may transition to the closed position to hold (e.g., substantially hold) the pressure in the inflatable member 104. In some examples, the active valve 118 may transition to the open position to transfer fluid back to the fluid reservoir 102, release pressure in the inflatable member 104 and/or allow a flow back to the inflatable member 104. In some examples, the active valve 118 may be used to hold (e.g., substantially hold) a partial inflation pressure.

Referring to FIG. 1B, the active valve 118 may include a diaphragm actuator 146 coupled to a protrusion 142. The diaphragm actuator 146 may include a metal-based material. In some examples, the diaphragm actuator 146 may be a thin, flexible piece (e.g., circular, or cylindrical portion) of a metal material (e.g., foil). In some examples, the protrusion 142 includes a metal-based material. In some examples, the protrusion 142 includes a metallic needle portion. In some examples, the protrusion 142 includes a tapered conical portion. The protrusion 142 may be coupled to a portion of the diaphragm actuator 146. In some examples, the protrusion 142 is welded to a surface (e.g., a central portion) of the diaphragm actuator 146. The active valve 118 includes a base plate 148 defining an inlet port 152 and an outlet port 150. When the active valve 118 is in the open position, fluid may be transferred from the inlet port 152 to the outlet port 150.

When the active valve 118 is in the closed position, fluid may be blocked from being transferred from the inlet port 152 to the outlet port 150. The base plate 148 may be a metal cylindrical portion. The inlet port 152 is an opening 151 (e.g., a tapered conical hole) configured to receive the protrusion 142 when the active valve 118 is in the closed position. The base plate 148 is coupled (e.g., welded) to the diaphragm actuator 146 such that the protrusion 142 is aligned with the opening 151 (or the inlet port 152). The active valve 118 includes a piezo element 140 coupled to the diaphragm actuator 146 via an epoxy material 144. In some examples, the piezo element 140 is configured to move (e.g., bend) when a voltage is applied to the piezo element 140.

In response to the piezo element 140 being activated (e.g., a voltage applied to the piezo element 140), the movement of the piezo element 140 may cause the diaphragm actuator 146 moves towards (e.g., bends towards) the base plate 148 such that the protrusion 142 moves into the opening 151 of the base plate 148 in a first direction A1 until the protrusion 142 contacts a portion 141 of the base plate 148 (defining the opening 151), thereby providing a metal-to-metal sealing. As shown in FIG. 1B, in response to the piezo element 140 being deactivated (e.g., a voltage being removed from the piezo element 140), the diaphragm actuator 146 moves away (e.g., bends away) from the base plate 148 in a direction A2 such that at least a portion of the protrusion 142 is disposed outside of the opening 151, thereby allowing the fluid to flow through the active valve 118.

Referring back to FIG. 1A, in some examples, the inflatable member 104 is an inflatable cuff member configured to be implemented around a urethra of a patient. In some examples, the inflatable member 104 is a penile inflation member (e.g., one or more inflatable cylinders) that may be implanted into the corpus cavernosum of the user. In some examples, the fluid reservoir 102 may be implanted in the abdomen or pelvic cavity of the user (e.g., the fluid reservoir 102 may be implanted in the lower portion of the user's abdominal cavity or the upper portion of the user's pelvic cavity). In some examples, at least a portion of the electronic pump assembly 106 may be implemented in the patient's body.

The fluid reservoir 102 may include a container having an internal chamber configured to hold or house fluid that is used to inflate the inflatable member 104. The volumetric capacity of the fluid reservoir 102 may vary depending on the size of the implantable device 100. In some examples, the volumetric capacity of the fluid reservoir 102 may be 3 to 150 cubic centimeters. In some examples, the fluid reservoir 102 is constructed from the same material as the inflatable member 104. In other examples, the fluid reservoir 102 is constructed from a different material than the inflatable member 104. In some examples, the fluid reservoir 102 contains a larger volume of fluid than the inflatable member 104.

The implantable device 100 may include a first conduit connector 103 and a second conduit connector 105. Each of the first conduit connector 103 and the second conduit connector 105 may define a lumen configured to transfer the fluid to and from the pump assembly 106. The first conduit connector 103 may be coupled to the electronic pump assembly 106 and the fluid reservoir 102 such that fluid can be transferred between the electronic pump assembly 106 and the fluid reservoir 102 via the first conduit connector 103. For example, the first conduit connector 103 may define a first lumen configured to transfer fluid between the electronic pump assembly 106 and the fluid reservoir 102. The first conduit connector 103 may include a single or multiple tube members for transferring the fluid between the electronic pump assembly 106 and the fluid reservoir 102.

The second conduit connector 105 may be coupled to the pump assembly 106 and the inflatable member 104 such that fluid can be transferred between the electronic pump assembly 106 and the inflatable member 104 via the second conduit connector 105. For example, the second conduit connector 105 may define a second lumen configured to transfer fluid between the electronic pump assembly 106 and the inflatable member 104. The second conduit connector 105 may include a single or multiple tube members for transferring the fluid between the electronic pump assembly 106 and the inflatable member 104. In some examples, the first conduit connector 103 and the second conduit connector 105 may include a silicone rubber material. In some examples, the electronic pump assembly 106 may be directly connected to the fluid reservoir 102.

The electronic pump assembly 106 may automatically transfer fluid between the fluid reservoir 102 and the inflatable member 104 without the user manually operating a pump (e.g., squeezing and releasing a pump bulb). The electronic pump assembly 106 that can monitor control and regulate a pressure within an inflatable member 104. The electronic pump assembly 106 may include a controller 114, one or more active valves 118, one or more pumps 120, and a pressure sensor 130 (or multiple pressure sensors). The controller 114 may control the pump(s) 120 and the active valve(s) 118 to move fluid between the inflatable member 104 and the fluid reservoir 102 to transition the inflatable member 104 between an inflation state and a deflation state. The pressure sensor 130 may monitor the pressure of the inflatable member 104. The controller 114 may receive pressure readings from the pressure sensor 130 and control the pump(s) 120 and the active(s) valves 118 to maintain and/or adjust the pressure of the inflatable member 104. The controller 114 may send control signals to the pump(s) 120 and the active valve(s) 118 to inflate or deflate the inflatable member 104. In some examples, the control of the inflation state and the deflation state is based on wireless signals 109 received from an external device 101 that is operated by the patient (and the detected pressure of the inflatable member 104 from the pressure sensor 130). For example, the patient may use the external device 101 to place the inflatable member 104 in an inflation or deflation state, which causes the external device 101 to send a wireless signal 109 to the controller 114.

The electronic pump assembly 106 may include a battery 116 configured to provide power to the controller 114 and other components on the electronic pump assembly 106. In some examples, the battery 116 is a non-rechargeable battery. In some examples, the battery 116 is a rechargeable battery. In some examples, the electronic pump assembly 106 (or a portion thereof) (or the controller 114) is configured to be connected to an external charger to charge the battery 116. In some examples, the electronic pump assembly 106 may define a charging interface that is configured to connect to the external charger. In some examples, the charging interface includes a universal serial bus (USB) interface configured to receive a USB charger. In some examples, the charging technology may be electromagnetic or Piezoelectric.

The electronic pump assembly 106 may include an antenna 112 configured to wirelessly transmit (and receive) wireless signals 109 from an external device 101. The external device 101 may be any type of component that can communicate with the electronic pump assembly 106. The external device 101 may be a computer, smartphone, tablet, pendant, key fob, etc. A user may use the external device 101 to control the implantable device 100. In some examples, the user may use the external device 101 to inflate or deflate the inflatable member 104. For example, in response to the user activating an inflation cycle using the external device 101 (e.g., selecting a user control on the external device 101), the external device 101 may transmit a wireless signal 109 to the electronic pump assembly 106 to initiate the inflation cycle (received via the antenna 112), where the controller 114 may control the active valve(s) 118 and the pump(s) 120 to inflate the inflatable member 104 to a target inflation pressure. In some examples, the controller 114 may cause the active valve 118 to a closed position and cause the pump(s) to operate to move fluid from the fluid reservoir 102 to the inflatable member 104.

In some examples, in response to the user activating a deflation cycle using the external device 101 (e.g., selecting a user control on the external device 101), the external device 101 may transmit a wireless signal 109 to the electronic pump assembly 106 to initiate the deflation cycle (received via the antenna 112), where the controller 114 may control the active valve(s) 118 (and, in some examples, the pump(s) 120) to transfer fluid from the inflatable member 104 to the fluid reservoir 102. For example, the controller 114 may control the active valve 118 to move to the open position to allow fluid to transfer from the inflatable member 104 to the fluid reservoir 102. In some examples, the controller 114 may control one or more pumps 120 to further move the fluid from the inflatable member 104 to the fluid reservoir 102 during the deflation cycle. In some examples, during the deflation cycle, fluid is transferred back until the pressure in the inflatable member 104 reaches a partial inflation pressure. In some examples, the controller 114 may automatically determine to initiate a deflation cycle, which causes the controller 114 to control the active valve(s) 118 (and, in some examples, the pump(s) 120) to transfer fluid back to the fluid reservoir 102.

The controller 114 may be any type of controller configured to control operations of the pump(s) 120 and the active valve(s) 118. In some examples, the controller 114 is a microcontroller. In some examples, the controller 114 includes one or more drivers configured to drive the pump(s) 120 and the active valve(s) 118. In some examples, the driver(s) are components separate from the controller 114. The controller 114 may be communicatively coupled to the active valve(s) 118, the pump(s) 120, and the pressure sensor(s) 130. In some examples, the controller 114 is connected to the active valve(s) 118, the pump(s) 120, and the pressure sensor(s) 130 via wired data lines. The controller 114 may include a processor 113 and a memory device 115.

The processor 113 may be formed in a substrate configured to execute one or more machine executable instructions or pieces of software, firmware, or a combination thereof. The processor 113 can be semiconductor-based—that is, the processors can include semiconductor material that can perform digital logic. The memory device 115 may store information in a format that can be read and/or executed by the processor 113. The memory device 115 may store executable instructions that when executed by the processor 113 cause the processor 113 to perform certain operations discussed herein. The controller 114 may receive data via the pressure sensor(s) 130 and/or the external device 101 and control the active valve(s) 118 and/or the pump(s) 120 by transmitting control signals to the active valve(s) 118 and/or the pump(s) 120.

The memory device 115 may store control parameters that can be set or modified by the user and/or physician using the external device 101. In some examples, the control parameters may include the target inflation pressure and/or the partial inflation pressure. In some examples, the target inflation pressure is a maximum (or desired) pressure allowable in the inflatable member 104. In some examples, the partial inflation pressure is a pressure threshold that can more closely mimic the natural experience and/or personal comfort of the user. A user or physician may update the control parameters using the external device 101, which can be communicated to the controller 114 via the antenna 112 and then updated in the memory device 115.

The external device 101 may communicate with the electronic pump assembly 106 over a network. In some examples, the network includes a short-range wireless network such as near field communication (NFC), Bluetooth, or infrared communication. In some examples, the network may include the Internet (e.g., Wi-Fi) and/or other types of data networks, such as a local area network (LAN), a wide area network (WAN), a cellular network, satellite network, or other types of data networks.

In some examples, the electronic pump assembly 106 includes a single pump 120 such as a pump 120-1. The pump 120-1 may be disposed in parallel with the active valve 118. In some examples, the electronic pump assembly 106 includes multiple pumps 120. For example, the pumps 120 include pump 120-1 and pump 120-2. In some examples, the pump 120-1 is disposed in a fluid passageway 125 that is used to fill the inflatable member 104 (e.g., during the inflation cycle). In some examples, the pump 120-2 is disposed in a fluid passageway 127 that is used to fill the inflatable member 104 (e.g., during the inflation cycle). In some examples, the pump 120-2 is disposed in parallel with the pump 120-1. The pump 120-1 may transfer fluid according to a first flow rate, and the pump 120-1 may transfer fluid according to a second flow rate. In some examples, the first flow rate is substantially the same as the second flow rate. In some examples, the first flow rate is different from the second flow rate.

In some examples, the pumps 120 may include more than two pumps 120 such as three, four, five, six, or greater than six pumps 120. For example, the pumps 120 may include a third pump in parallel with the pump 120-2, a fourth pump in parallel with the third pump, and so forth. In some examples, the pumps 120 may include one or more pumps 120 in series with one or more other pumps 120. For example, one or more pumps 120 may be in series with the pump 120-1. In some examples, one or more pumps 120 may be in series with the pump 120-2.

Each pump 120 is an electronically-controlled pump. Each pump 120 may be electronically-controlled by the controller 114. For example, each pump 120 may be connected to the controller 114 and may receive a signal to actuate a respective pump 120. A pump 120 may be unidirectional in which the pump 120 can transfer fluid from the fluid reservoir 102 to the inflatable member 104 (or from the inflatable member 104 to the fluid reservoir 102). In some examples, a pump 120 is bidirectional in which the pump 120 can transfer fluid from the fluid reservoir 102 to the inflatable member 104 and from the inflatable member 104 to the fluid reservoir 102. In some examples, the pumps 120 are either unidirectional or bidirectional. In some examples, the pumps 120 include a combination of one or more unidirectional pumps and one or more bidirectional pumps.

In some examples, the pump 120 is an electromagnetic pump that moves the fluid between the fluid reservoir 102 and the inflatable member 104 using electromagnetism. With respect to an electromagnetic pump, a magnetic fluid is set at angles to the direction the fluid moves in, and a current is passed through it.

In some examples, the pump 120 is a piezoelectric pump. In some examples, a piezoelectric pump may be a diaphragm micropump that uses actuation of a diaphragm to drive a fluid. In some examples, a piezoelectric pump may include one or more piezo pumps (e.g., piezo elements), which may be implemented by a substrate layer (e.g., a single substrate layer) of high-voltage piezo elements or may be implemented by multiple substrate layers (e.g., stacked substrate layers) of low-voltage piezo elements. In some examples, the pump 120 includes a plurality of micro-pumps (e.g., piezoelectrically-driven micro-pumps) disposed on one or more substrates (e.g., wafer(s)). In some examples, the micro-pumps include a silicon-based material. In some examples, the micro-pumps include a metal (e.g., steel) based material. In some examples, the pump 120 is non-mechanical (e.g., without moving parts).

In some examples, in the case of multiple pumps 120, each pump 120 may be a pump of the same type (e.g., all pumps 120 are electromagnetic pumps or all pumps 120 are piezoelectric pumps). In some examples, one or more pumps 120 are different from one or more other pumps 120. For example, pumps 120 may include different types of piezoelectric pumps or the pumps 120 may include different types of electromagnetic pumps. The pump 120-1 may be a piezoelectric pump having a first number of micro-pumps, and the pump 120-2 may be a piezoelectric pump having a second number of micro-pumps (where the second number is different from the first number). The pump 120-1 may be an electromagnetic pump, and the pump 120-2 may be a piezoelectric pump.

A pump 120 may include one or more passive check valves. The passive check valve(s) may assist with maintaining pressure in the inflatable member 104. In some examples, a pump 120 may include a single passive check valve. In some examples, the pump 120 may include multiple passive check valves such as two passive check values or more than two passive check valves. The passive check valve(s) of a respective pump 120 may not be directly controlled by the controller 114, but rather based on the pressure between the inflatable member 104 and the fluid reservoir 102. The passive check valve(s) may transition between an open position (in which fluid is permitted to flow through the passive check valve(s)) and a closed position (in which fluid is prevented from flowing through the passive check valve(s)). In some examples, the passive check valve(s) transitions to the closed position in response to positive pressure between the inflatable member 104 and the fluid reservoir 102. In some examples, the passive check valve(s) transition to the open position in response to negative pressure between the inflatable member 104 and the fluid reservoir 102.

In some examples, the use of two parallel pumps (e.g., pump 120-1, pump 120-2) (or more than two parallel pumps 120) may increase the amount of fluid that can be transferred to the inflatable member 104. In some examples, the pumps 120 may operate out of phase from each other in order to increase the efficiency of the electronic pump assembly 106. Two parallel pumps (e.g., pump 120-1, pump 120-2) operating at out of phase (e.g., 180 degrees of out of phase) from each other may allow the output pressure of the pump 120-1 to improve the valve closure of the pump 120-2, thereby improving the overall performance (and vice versa). The use of parallel pumps 120 operating out of phase from each other may allow the pumps 120 to operate at lower frequencies, which can reduce power (thereby extending battery life). Furthermore, a smoother flow rate may also be achieved resulting in less vibration and an improved patient experience. As indicated above, one or more pumps 120 may be in series with one or more parallel pumps 120. For example, an additional pump 120 may be in series with the pump 120-1, and/or an additional pump 120 may be in series with the pump 120-2. Serial pump operation may enable doubling of the pressure when two similar-performing pumps 120 are utilized. In some examples, two or more serially-disposed pumps 120 may be operated at the same phase.

In some examples, the electronic pump assembly 106 includes a hermetic enclosure 108 that encloses the components of the electronic pump assembly 106. A hermetic enclosure 108 may be an air-tight (or substantially air-tight) container. The hermetic enclosure 108 may include one or more metal-based materials. In some examples, the hermetic enclosure 108 is a Titanium container. In some examples, the only material in contact with the patient is Titanium. In some examples, the hermetic enclosure 108 includes one or more non-metal-based materials (e.g., ceramic). In some examples, a portion of the hermetic enclosure 108 is a metal-based material and a portion of the hermetic enclosure 108 is a non-metal-based material. In some examples, the hermetic enclosure 108 defines a feedthrough (e.g., a hermetic feedthrough, an electrical feedthrough, a feedthrough connector, etc.) to receive/transmit wireless signals from/to the external device 101. In some examples, the feedthrough includes a metal-based material and an insulator-based material (e.g., ceramic).

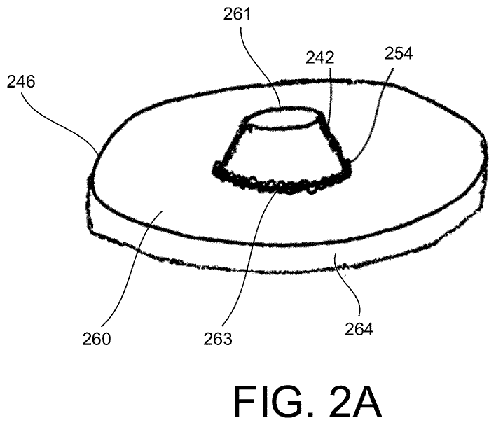

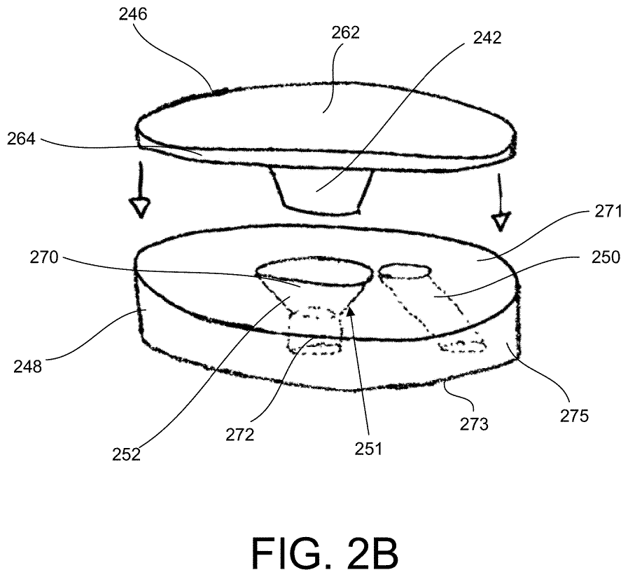

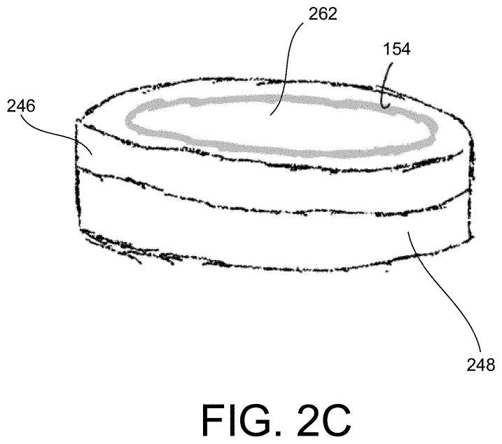

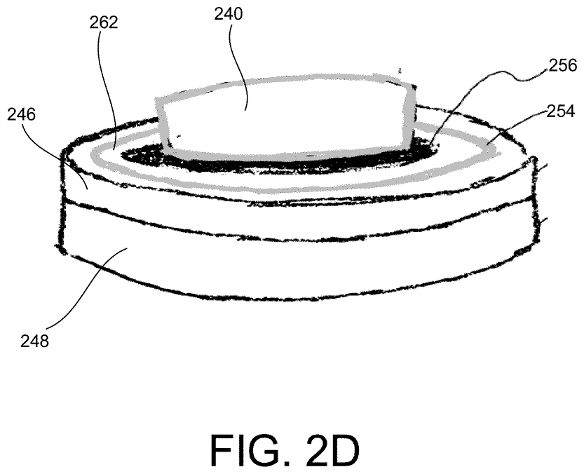

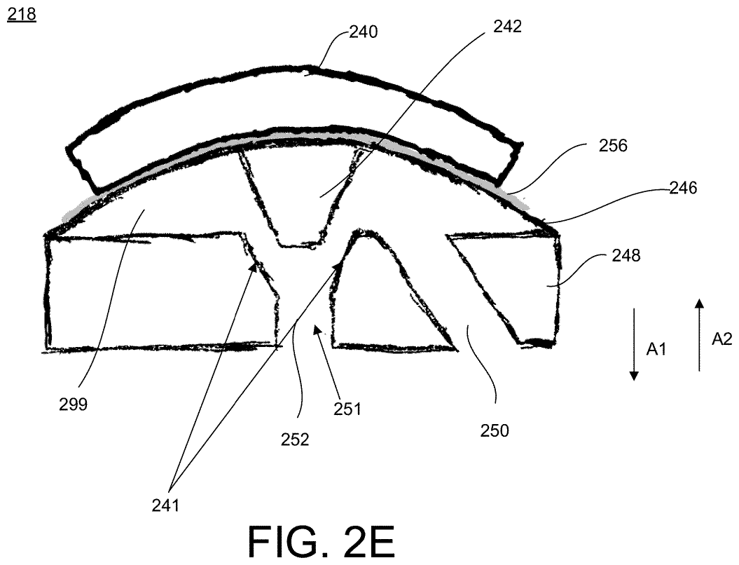

FIGS. 2A through 2E illustrate an active valve 218 according to an aspect. As shown in FIG. 2A, the active valve 218 includes a diaphragm actuator 246 coupled to a protrusion 242. The diaphragm actuator 246 may include a metal-based material. In some examples, the diaphragm actuator 246 may be a thin, flexible piece of a metal material (e.g., foil). In some examples, the diaphragm actuator 246 includes a cylindrical portion. The diaphragm actuator 246 includes a first surface 260 and a second surface 262 (shown in FIG. 2B), where the distance between the first surface 260 and the second surface 262 defines a thickness of the diaphragm actuator 246. A sidewall 264 extends between the first surface 260 and the second surface 262. The first surface 260 may have a disc shape. The second surface 262 may have a disc shape.

The protrusion 242 may include a metal-based material. In some examples, the protrusion 242 is a needle portion (e.g., a metallic needle portion). In some examples, the protrusion 242 includes a conical portion. In some examples, the protrusion 242 includes a tapered conical portion. The protrusion 242 includes a first end portion 261 and a second end portion 263. The first end portion 261 may have a smaller size (e.g., diameter) than a size (e.g., diameter) of the second end portion 263.

The protrusion 242 is coupled to the diaphragm actuator 246. In some examples, the protrusion 142 is coupled to the diaphragm actuator 246 via a welded portion 254 (e.g., the protrusion 142 is welded to the diaphragm actuator 246. The second end portion 263 of the protrusion 242 may be coupled to the first surface 260 of the diaphragm actuator 246. In some examples, the second end portion 263 of the protrusion 242 is coupled to a central portion of the first surface 260 of the diaphragm actuator 246.

As shown in FIG. 2B, the active valve 218 includes a base plate 248. The base plate 248 may include a metal-based material. In some examples, the base plate 248 includes a cylindrical portion (e.g., metallic cylindrical portion). The base plate 248 may include a first surface 271 and a second surface 273, where a distance between the first surface 271 and the second surface 273 defines a thickness of the base plate 248. The base plate 248 includes a sidewall 275 that extends between the first surface 271 and the second surface 273. In some examples, the base plate 248 may have a diameter that is the same (or substantially the same (e.g., within 1 or 2 millimeters)) as a diameter of the diaphragm actuator 246.

The base plate 248 may define an inlet port 252 and an outlet port 250. When the active valve 218 is in the open position (as shown in FIG. 2E), fluid may be transferred from the inlet port 252 to the outlet port 250 via a chamber 299 (shown in FIG. 2E). When the active valve 218 is in the closed position, fluid may be blocked from being transferred from the inlet port 152 to the outlet port 150 (e.g., blocked by the protrusion 242). The inlet port 252 is an opening 251 (e.g., a hole) that extends through the thickness of the base plate 248. In some examples, the thickness of the base plate 248 is greater than the length of the protrusion 242, where the length of the protrusion 242 may be the distance between the first end portion 261 and the second end portion 263. The outlet port 250 is an opening (e.g., a hole) that extends through the thickness of the base plate 248. In some examples, the outlet port 250 has a shape that is different from the shape of the inlet port 252. In some examples, the outlet port 250 is not tapered.

In some examples, the opening 251 includes a tapered cylindrical portion 270 configured to receive the protrusion 242 when the active valve 218 is in the closed position. In some examples, the protrusion 242 (e.g., the length of the protrusion 242) is disposed in the tapered cylindrical portion 270 when the active valve 218 is in the closed position. In some examples, the opening 251 includes a non-tapered cylindrical portion 272. In some examples, the protrusion 242 is not disposed in the non-tapered cylindrical portion 272 when the active valve 218 is in the closed position.

As shown in FIG. 2C, the base plate 248 is coupled (e.g., welded) to the diaphragm actuator 246 such that the protrusion 242 is aligned with the opening 251 (or the inlet port 252). In some examples, a weld ring 154 is formed on the second surface 262 of the diaphragm actuator 246. As shown in FIG. 2D, the active valve 218 includes a piezo element 240 coupled to the second surface 262 of the diaphragm actuator 246 via an epoxy material 256.

FIG. 2E illustrates the active valve 218 in the open position. In response to the piezo element 240 being activated (e.g., a voltage applied to the piezo element 240), the diaphragm actuator 246 moves towards (e.g., bends towards) the base plate 248 such that the protrusion 242 moves into the opening 251 of the base plate 248 in a first direction A1 until the protrusion 242 contacts a portion 241 of the base plate 148 (defining the opening 151), thereby providing a metal-to-metal sealing. As shown in FIG. 2E, in response to the piezo element 240 being deactivated (e.g., a voltage being removed from the piezo element 240), the diaphragm actuator 246 moves away (e.g., bends away) from the base plate 248 in a direction A2 such that at least a portion of the protrusion 242 is disposed outside of the opening 251, thereby allowing the fluid to flow through the active valve 218.

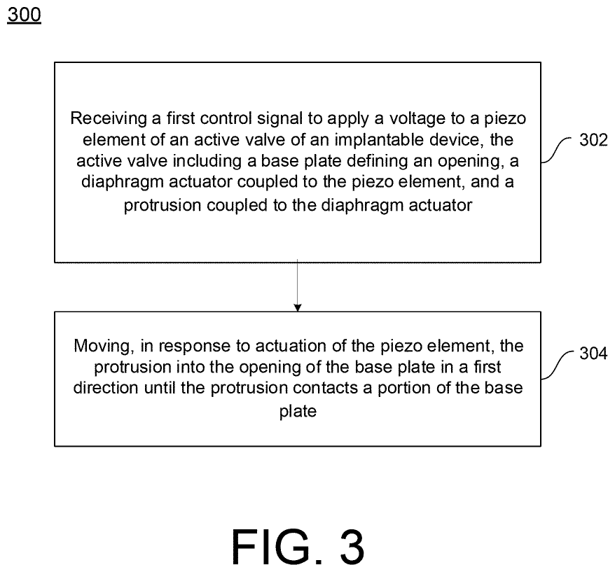

FIG. 3 illustrates a flow chart 300 depicting example operations of a method of actuating an active valve of an electronic pump assembly of an inflatable member. Although the operations of FIG. 3 are described with reference to the active valve 118 of FIGS. 1A and 1B, the example operations of the flow chart 300 may be performed by any of the active valves and/or electronic pump assemblies discussed herein.

Operation 302 includes receiving a first control signal to apply a voltage to a piezo element 140 of an active valve 118 of an implantable device 100, the active valve 118 including a base plate 148 defining an opening 150, a diaphragm actuator 146 coupled to the piezo element 140, and a protrusion 142 coupled to the diaphragm actuator 146. Operation 304 includes moving, in response to actuation of the piezo element 140, the protrusion 142 into the opening 151 of the base plate 148 in a first direction A1 until the protrusion 142 contacts a portion 141 of the base plate 148.

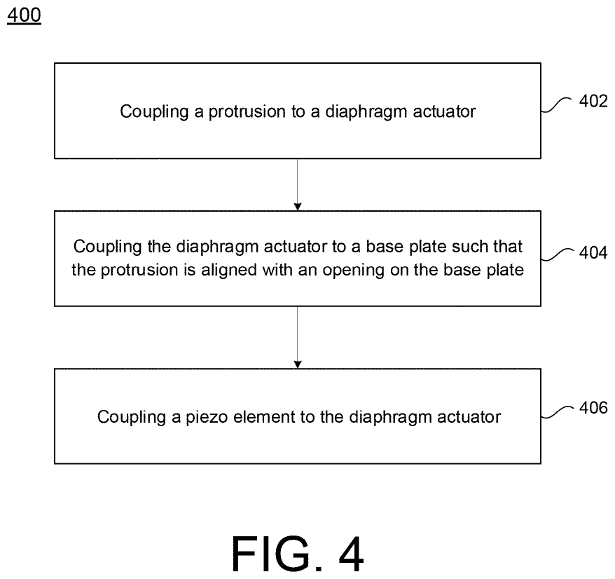

FIG. 4 illustrates a flow chart 400 depicting example operations of a method of manufacturing an active valve of an electronic pump assembly of an inflatable member. Although the operations of FIG. 4 are described with reference to the active valve 218 of FIGS. 2A through 2E, the example operations of the flow chart 400 may be performed by any of the active valves and/or electronic pump assemblies discussed herein.

Operation 402 includes coupling a protrusion 242 to a diaphragm actuator 246. As shown in FIG. 2A, the protrusion 242 is coupled to the diaphragm actuator 246 via a welding (e.g., a welded bond). Operation 404 includes coupling the diaphragm actuator 246 to a base plate 248 such that the protrusion 242 is aligned with an opening 251 on the base plate 248. As shown in FIGS. 2B and 2C, the diaphragm actuator 246 is moved towards and contacts the base plate 248 in which the protrusion 242 is aligned with the opening 251, and the diaphragm actuator 246 is coupled to the base plate (via welding). Operation 406 includes coupling a piezo element 240 to the diaphragm actuator 246. As shown in FIG. 2D, the piezo element 240 is coupled to the diaphragm actuator 246 using an epoxy material 256.

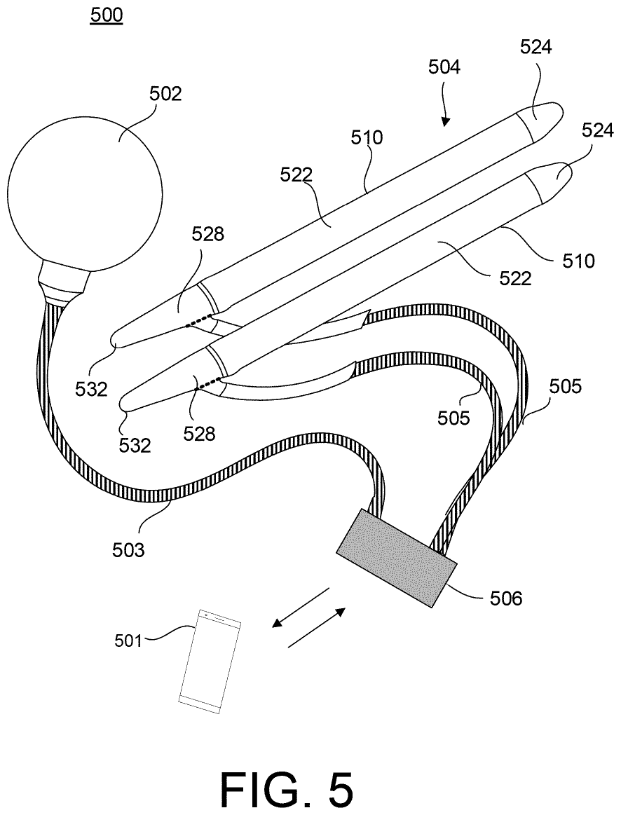

FIG. 5 schematically illustrates an inflatable penile prosthesis 500 having an electronic pump assembly 506 according to an aspect. The electronic pump assembly 506 may include any of the features of the electronic pump assembly discussed herein, including active valve 118 of FIGS. 1A and 1B or active valve 218 of FIGS. 2A through 2E. The inflatable penile prosthesis 500 may include an inflatable member 504 (e.g., a pair of inflatable cylinders 510), and the inflatable cylinders 510 are configured to be implanted in a penis. For example, one of the inflatable cylinders 510 may be disposed on one side of the penis, and the other inflatable cylinder 510 may be disposed on the other side of the penis. Each inflatable cylinder 510 may include a first end portion 524, a cavity or inflation chamber 522, and a second end portion 528 having a rear tip 532.

At least a portion of the electronic pump assembly 506 may be implanted in the patient's body. A pair of conduit connectors 505 may attach the electronic pump assembly 506 to the inflatable cylinders 510 such that the electronic pump assembly 506 is in fluid communication with the inflatable cylinders 510. Also, the electronic pump assembly 506 may be in fluid communication with a fluid reservoir 502 via a conduit connector 503. The fluid reservoir 502 may be implanted into the user's abdomen. The inflation chamber 522 of the inflatable cylinder 510 may be disposed within the penis. The first end portion 524 of the inflatable cylinder 510 may be at least partially disposed within the crown portion of the penis. The second end portion 528 may be implanted into the patient's pubic region PR with the rear tip 532 proximate to the pubic bone PB.

In order to implant the inflatable cylinders 510, the surgeon first prepares the patient. The surgeon often makes an incision in the penoscrotal region, e.g., where the base of the penis meets with the top of the scrotum. From the penoscrotal incision, the surgeon may dilate the patient's corpus cavernosum to prepare the patient to receive the inflatable cylinders 510. The corpus cavernosum is one of two parallel columns of erectile tissue forming the dorsal part of the body of the penis, e.g., two slender columns that extend substantially the length of the penis. The surgeon will also dilate two regions of the pubic area to prepare the patient to receive the second end portion 528. The surgeon may measure the length of the corpora cavernosum from the incision and the dilated region of the pubic area to determine an appropriate size of the inflatable cylinders 510 to implant.

After the patient is prepared, the inflatable penile prosthesis 500 is implanted into the patient. The tip of the first end portion 524 of each inflatable cylinder 510 may be attached to a suture. The other end of the suture may be attached to a needle member (e.g., Keith needle). The needle member is inserted into the incision and into the dilated corpus cavernosum. The needle member is then forced through the crown of the penis. The surgeon tugs on the suture to pull the inflatable cylinder 510 into the corpus cavernosum. This is done for each inflatable cylinder 510 of the pair. Once the inflation chamber 522 is in place, the surgeon may remove the suture from the tip. The surgeon then inserts the second end portion 528. The surgeon inserts the rear end of the inflatable cylinder 510 into the incision and forces the second end portion 528 toward the pubic bone PB until each inflatable cylinder 510 is in place.

A user may use an external device 501 to control the inflatable penile prosthesis 500. In some examples, the user may use the external device 501 to inflate or deflate the inflatable cylinders 510. For example, in response to the user activating an inflation cycle using the external device 501, the external device 501 may transmit a wireless signal to the electronic pump assembly 506 to initiate the inflation cycle to transfer fluid from the fluid reservoir 502 to the inflatable cylinders 510. In some examples, in response to the user activating a deflation cycle using the external device 501, the external device 501 may transmit a wireless signal to the electronic pump assembly 506 to initiate the deflation cycle to transfer fluid from the inflatable cylinders 510 to the fluid reservoir 502. In some examples, during the deflation cycle, fluid is transferred back until the pressure in the inflatable cylinders 510 reaches a partial inflation pressure.

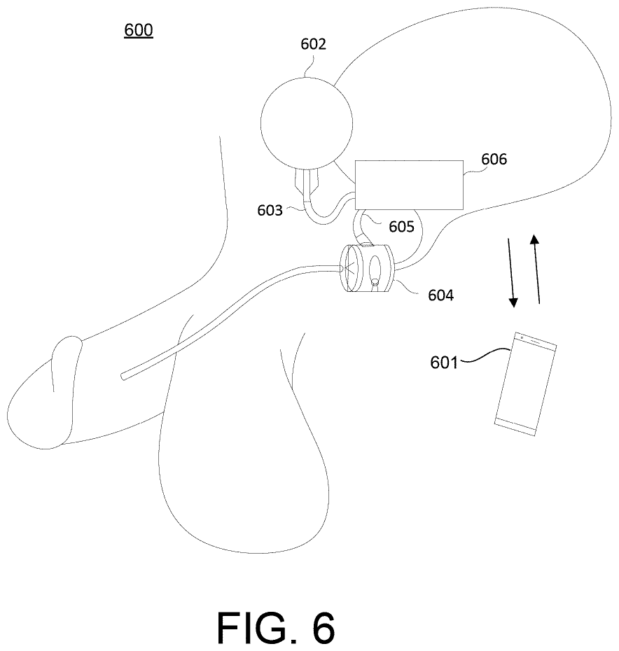

FIG. 6 illustrates a urinary control device 600 having an electronic pump assembly 606 according to an aspect. The electronic pump assembly 606 may include any of the features of the electronic pump assembly discussed herein, including active valve 118 of FIGS. 1A and 1B or active valve 218 of FIGS. 2A through 2E. The urinary control device 600 includes a pump assembly 606, a fluid reservoir 602, and a cuff 604.

The fluid reservoir 602 may be a pressure-regulating inflation balloon or element. The fluid reservoir 602 is in operative fluid communication with the cuff 604 via one or more tube members 603, 605. The fluid reservoir 602 is constructed of polymer material that is capable of elastic deformation to reduce fluid volume within the fluid reservoir 602 and push fluid out of the fluid reservoir 602 and into the cuff 604. However, the material of the fluid reservoir 602 can be biased or include a shape memory construct adapted to generally maintain the fluid reservoir 602 in its expanded state with a relatively constant fluid volume and pressure. In some examples, this constant level of pressure exerted from the fluid reservoir 602 to the cuff 604 will keep the cuff 604 at a desired inflated state when open fluid communication is provided between the fluid reservoir 602 and the cuff 604. This is largely due to the fact that only a small level of fluid displacement is required to inflate or deflate the cuff 604. In some examples, the fluid reservoir 602 is implanted into the abdominal space.

A user may use an external device 601 to control the urinary control device 600. In some examples, the user may use the external device 601 to inflate or deflate the cuff 604. For example, in response to the user activating an inflation cycle using the external device 601, the external device 601 may transmit a wireless signal to the electronic pump assembly 606 to initiate the inflation cycle to transfer fluid from the fluid reservoir 602 to the cuff 604 (e.g., by opening an active valve where the pressure in the fluid reservoir 602 causes the fluid to move through the active valve to the cuff 604). In some examples, in response to the user activating a deflation cycle using the external device 601, the external device 601 may transmit a wireless signal to the electronic pump assembly 606 to initiate the deflation cycle to transfer fluid from the cuff 604 to the fluid reservoir 602.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An active valve for an implantable device, the active valve comprising:
a base plate defining an opening;
a piezo element;
a diaphragm actuator coupled to the piezo element; and
a protrusion coupled to the diaphragm actuator,
the diaphragm actuator, in response to the piezo element being activated, is configured to move the protrusion into the opening in a first direction until the protrusion contacts a portion of the base plate.

2. The active valve of claim 1, wherein the protrusion includes a tapered conical portion.

3. The active valve of claim 1, wherein the protrusion includes a metal-based material.

4. The active valve of claim 1, wherein the opening includes a tapered conical hole.

5. The active valve of claim 1, wherein the diaphragm actuator includes a metal-based material.

6. The active valve of claim 1, wherein the diaphragm actuator is coupled to the base plate.

7. The active valve of claim 1, wherein the diaphragm actuator includes a first surface and a second surface, the piezo element being coupled to the first surface of the diaphragm actuator, the protrusion being coupled to the second surface of the diaphragm actuator.

8. The active valve of claim 1, wherein the diaphragm actuator includes a metal-based material.

9. The active valve of claim 1, wherein at least a portion of the protrusion is disposed outside of the opening of the base plate in response to the piezo element not being actuated.

10. An implantable device comprising:
a fluid reservoir configured to hold fluid;
an inflatable member; and
an electronic pump assembly including a controller and an active valve, the active valve including:
a base plate defining an opening;
a piezo element;
a diaphragm actuator coupled to the piezo element; and
a protrusion coupled to the diaphragm actuator,
the controller is configured to activate the piezo element to move the protrusion into the opening in a first direction until the protrusion contacts a portion of the base plate.

11. The implantable device of claim 10, wherein the controller is configured to controller is configured to deactivate the piezo element to move at least a portion of the protrusion out of the opening.

12. The implantable device of claim 10, wherein the protrusion includes a metallic needle portion, the metallic needle portion including a tapered conical portion.

13. The implantable device of claim 12, wherein the base plate includes a metal-based material, the opening on the base plate including a tapered conical hole configured to receive the tapered conical portion.

14. The implantable device of claim 10, wherein the diaphragm actuator is welded to the base plate.

15. The implantable device of claim 10, wherein the piezo element is coupled to the diaphragm actuator with an epoxy-based material.

16. The implantable device of claim 10, wherein the opening is an inlet port configured to receive fluid, the base plate defining an outlet port to output fluid.

17. The implantable device of claim 16, wherein the inlet port is coupled to the inflatable member and the outlet port is coupled to the fluid reservoir.

18. A method for actuating an active valve of an implantable device, the method comprising:
receiving a first control signal to apply a voltage to a piezo element of an active valve of an implantable device, the active valve including a base plate defining an opening, a diaphragm actuator coupled to the piezo element, and a protrusion coupled to the diaphragm actuator; and
moving, in response to actuation of the piezo element, the protrusion into the opening of the base plate in a first direction until the protrusion contacts a portion of the base plate.

19. The method of claim 18, further comprising:
receiving a second control signal to not apply the voltage to the piezo element of the active valve; and
moving, in response to the second control signal, the protrusion in a second direction such that at least a portion of the protrusion is disposed outside of the opening of the base plate.

20. The method of claim 18, further comprising:
forming a metal-to-metal seal with the protrusion and the base plate.
